(19) United States
(12) Patent Application Publication
(10) Pub. No.: US 2021/0406394 A1
(43) Pub. Date: Dec. 30, 2021

(54) PRE-REGISTRATION OF SECURE TRAVEL INFORMATION

(71) Applicant: Bank of America Corporation, Charlotte, NC (US)

(72) Inventors: Albena N. Fairchild, Indian Trail, NC (US); Daniel Doherty, Huntersville, NC (US)

(21) Appl. No.: 16/912,138

(22) Filed: Jun. 25, 2020

(51) Int. Cl. G06F 21/62; G16H 10/60; G06F 21/60; H04L 29/06

(52) U.S. Cl. CPC G06F 21/6245; G16H 10/60; H04L 63/0838; H04L 63/0861; G06F 21/602

(57) ABSTRACT

A blockchain-based travel security system is provided. The system increases travel security. The system includes an electronically stored user's itinerary file with a user's pre-registered travel itinerary. The itinerary file can be stored on a blockchain. The system also includes an electronically stored user's medical history file stored on the blockchain. A single trusted information source provides the itinerary file and the medical history file to the blockchain. The system includes a dual track information storage and retrieval system which is configured to store the itinerary file and to store the recent medical history file. The dual track information storage and retrieval system comprises a first track related to the user's recent medical history file and a second track related to the itinerary file.

### FIELD OF TECHNOLOGY

This disclosure relates to blockchain systems.

### BACKGROUND

A blockchain is a distributed database of records or public ledger of all transactions or digital events that have been executed and shared among participants. In some block chains, each transaction or digital event in the public ledger is verified by a majority of participants included in the system. In other block chains, each transaction or digital event in the public ledger is verified by a single trusted source. Once a transaction or digital event is executed, it can never be erased. Therefore, the blockchain contains a certain and verifiable record of every single transaction.

Because of the unique characteristics specific to blockchain technology—i.e., no central database and the inability to erase a transaction—blockchain technology has the ability to revolutionize the digital world by enabling a distributed record of every online transaction that can be verified at any time in the future. Blockchain technology does not compromise the privacy of digital assets and the parties involved because the blocks, included on the chain, either do not include private data or include an encrypted version of private data. The distributed consensus and the anonymity are two important characteristics of blockchain technology.

One area of need for increasing security while maintaining anonymity involves travel. Recently, the importance of ensuring secure travel has increased.

For example, personal security issues along with grave recent health issues have highlighted the need for improving travel security.

It would be desirable to leverage blockchain technology to provide systems and methods that increase travel security.

Such systems and methods would preferably harness the properties of blockchain technology to secure travel.

It would be further desirable to utilize blockchain technology in connection with trusted source components to further increase travel security.

### SUMMARY OF THE DISCLOSURE

Apparatus and methods for increasing travel security are provided. One method, according to the embodiments, for increasing travel security involves a dual track information storage and retrieval system.

The method may include electronically storing on a blockchain an itinerary file. The itinerary file may include a user's travel itinerary for an upcoming trip. Such an itinerary may include names of travel service providers, destinations, restaurants, financial service locations, etc. Preferably, all of this information may be stored on the blockchain.

It should be noted that in embodiments of the invention that utilize storage on a blockchain—the writing to the blockchain preferably utilizes a single trusted source of information. In some embodiments, the user or other relevant party may only be able to write to the single trusted source of information using one more user biometric characteristic(s) as a security measure. Accordingly, a user, or other relevant entity with user permission, may be required to enter a user biometric prior to writing information to the single trusted source.

It should be noted as well that the viewing and/or access of the information on the blockchain may preferably be open to the travel services providers as well as other entities. In certain embodiments, the blockchain will only be open on a need to know basis—i.e., an airline may not have read access for hotel information and a hotel may not have read access for information regarding car rental, etc.

The method may further include electronically storing on the blockchain a medical history file associated with the user. Medical history stored on the file may include vaccinations, antibody coun

In addition, some embodiments may include authenticating the itinerary file and providing, using a single trusted information source, the itinerary file to the blockchain. Further certain embodiment may include authenticating the medical history file and providing, using the single trusted information source, the medical history file to the blockchain.

In some embodiments, the dual track information retrieval and storage system may operate to store the authenticated itinerary file and an authenticated recent medical history file.

In certain embodiments, the dual track information storage and retrieval system may utilize a first track for storing and retrieving the user's recent medical history, and a second track for storing and retrieving the itinerary file.

The authenticating each of the itinerary file and the medical history file may include using the single trusted information source to verify a biometric parameter associated with the user preferably prior to writing to the blockchain.

### BRIEF DESCRIPTION OF THE DRAWINGS

The objects and advantages of the invention will be apparent upon consideration of the following detailed description, taken in conjunction with the accompanying drawings, in which like reference characters refer to like parts throughout, and in which:

FIG. 1 shows an illustrative diagram in accordance with principles of the disclosure;

FIG. 2 shows another illustrative diagram in accordance with principles of the disclosure;

FIG. 3 shows an illustrative timing diagram in accordance with principles of the disclosure;

FIG. 4 shows another illustrative timing diagram in accordance with principles of the disclosure;

FIG. 5 shows an illustrative flow diagram in accordance with the principles of the disclosure; and

FIG. 6 shows still another illustrative diagram in accordance with the principles of the disclosure.

### DETAILED DESCRIPTION

A system for increasing travel security is provided. The system may include an electronically stored itinerary file. The itinerary file may include a user's preferably pre-registered travel itinerary. The travel itinerary may be stored on a blockchain.

The system may also include an electronically stored medical history file. The medical history file may include a user's recent medical history. The medical history file may be stored on the blockchain.

In addition, the system may include a single trusted information source. The single trusted information source may provide information to the blockchain. For example, the single trusted information source may provide the itinerary file and the medical history file to the blockchain.

In certain embodiments, the system may include a dual track information storage and retrieval system. The dual track information storage and retrieval system may be configured to store the itinerary information and to store the recent medical history.

The dual track information storage and retrieval system may include a first track related to the user's recent medical history. The dual track information storage and retrieval system may include a second track related to the user's travel itinerary.

In certain embodiments, the first track may also include a flag indicating the presence or non-presence of a preferably pre-determined plurality of antibodies related to a pre-determined virus. In some embodiments, the flag, or multiple flags, may be used to indicate any type of medical information. Such a flag or flags may be written to the blockchain by the single trusted source of information. Then, such information may be retrieved or checked by entities with view (alternatively referred to as “read”) access to the blockchain.

In some embodiments, the first track, the second track, both tracks or neither track may include a first-in first-out (FIFO) buffer, or other suitable buffer, in the storage system. Such a buffer may selectively delete a portion of the recent medical history that has been stored for more than a pre-determined amount of time.

In some embodiments, the first track, the second track, both tracks or neither track may further include a storage system that deletes all the recent medical history that has been stored for a pre-determined amount of time.

The first track may, in certain embodiments, include a deletion system. The deletion system may be the apparatus for overwriting or otherwise deleting the medical history that has been stored for more than a pre-determined amount of time.

In some embodiments, the deletion system can selectively delete all or portions of the recent medical history that has been stored for more than a pre-determined amount of time.

In certain embodiments, the deletion system can delete the medical history that has been stored for more than a 24-hour, or other suitable, period of time.

The second track may include an itinerary adjustment system. The itinerary adjustment system may enable real-time adjustments to the itinerary. Such adjustments may be made in response to a communication from the single trusted information source or in response to some other suitable communication.

A method for increasing travel security is also provided. The method may include electronically storing, in response to a communication from a single trusted information source, on a blockchain an itinerary file comprising a user's travel itinerary.

The electronically storing on the blockchain may include electronically storing a medical history file associated with the user.

In certain embodiments, the electronically storing may include authenticating the itinerary file. The method may also include providing, using the single trusted information source, the itinerary file to the blockchain.

In some embodiments, the method may include authenticating the medical history file and providing, using the single trusted information source, the medical history file to the blockchain.

The system for use with the method may include a dual track information storage and retrieval system, as set forth in detail above.

Certain methods for increasing travel security according to the invention may include electronically storing on a blockchain an itinerary file comprising a user's travel itinerary and electronically storing on the blockchain a medical history file associated with the user. The electronic storing on the blockchain may operate to store only information that is received from a trusted information source.

The method may also include authenticating the itinerary file and providing, using a single trusted information source, the itinerary file to the blockchain, authenticating the medical history file and providing, using the single trusted information source, the medical history file to the blockchain.

In certain embodiments of the method, the method may leverage a system that includes a dual track information storage and retrieval system. The dual track information retrieval and storage system may operate to store the itinerary information. The dual track information retrieval and storage system may operate to authenticate recent medical history.

The dual track information storage and retrieval system may include a first track for storing and retrieving the user's recent medical history and a second track for storing and retrieving the user's travel itinerary.

The authenticating each of the itinerary file and the authenticating the medical history file may include using the single trusted information source to verify a biometric parameter associated with the user.

In certain embodiments, each of the authenticating the itinerary file and the authenticating the medical history file may include using the single trusted information source to verify a one-time password associated with a user device.

In certain embodiments, each of the authenticating the itinerary file and the authenticating the medical history file may include using the single trusted information source to verify a registered token associated with a user device that provided, or continues to provide, one or both of the files.

Illustrative embodiments of apparatus and methods in accordance with the principles of the invention will now be described with reference to the accompanying drawings, which form a part hereof. It is to be understood that other embodiments may be utilized and structural, functional and procedural modifications may be made without departing from the scope and spirit of the present invention.

The drawings show illustrative features of apparatus and methods in accordance with the principles of the invention. The features are illustrated in the context of selected embodiments. It will be understood that features shown in connection with one of the embodiments may be practiced in accordance with the principles of the invention along with features shown in connection with another of the embodiments.

Apparatus and methods described herein are illustrative. Apparatus and methods of the invention may involve some or all of the features of the illustrative apparatus and/or some or all of the steps of the illustrative methods. The steps of the methods may be performed in an order other than the order shown or described herein. Some embodiments may omit steps shown or described in connection with the illustrative methods. Some embodiments may include steps that are not shown or described in connection with the illustrative methods, but rather shown or described in a different portion of the specification.

One of ordinary skill in the art will appreciate that the steps shown and described herein may be performed in other than the recited order and that one or more steps illustrated may be optional. The methods of the above-referenced embodiments may involve the use of any suitable elements, steps, computer-executable instructions, or computer-readable data structures. In this regard, other embodiments are disclosed herein as well that can be partially or wholly implemented on a computer-readable medium, for example, by storing computer-executable instructions or modules or by utilizing computer-readable data structures.

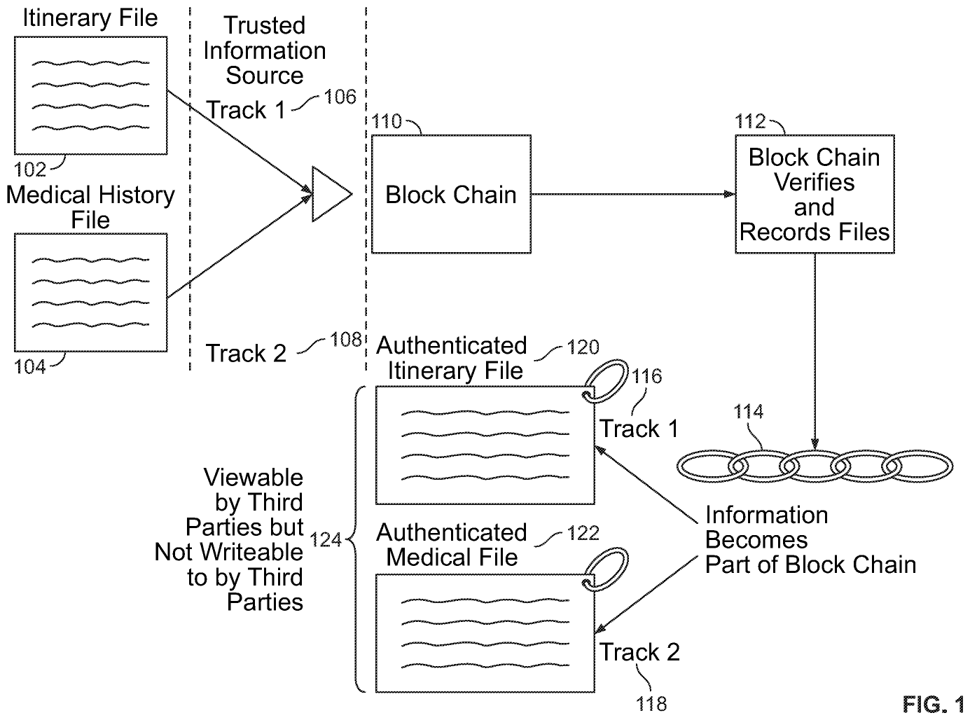

FIG. 1 shows an illustrative diagram in accordance with principles of the disclosure. FIG. 1 shows an itinerary file 102 and a medical history file 104.

Itinerary file 102 may be received from a user. Itinerary file 102 may include a user's travel itinerary. Information in itinerary file 102 may include travel services providers'—e.g., hotels, airlines, car rental companies, etc.—and their respective contact information. It may include times and dates of departure and arrival.

Medical history file 104 may include a current medical history of the user/traveler. For example, medical history file 104 may include information as to vaccinations. Medical history file 104 may include information as to antibody levels for a particular pathogen, history of baseline vital signs such as blood sugar, blood pressure, blood oxygenation level, pulse rate, or any other suitable medical information. Medical history file 104 may include any relevant historical and/or current medical information regarding the user.

Itinerary file 102 may be manipulated along a first track 106 (track 1). Medical history file 104 may be manipulated along a second track 108 (track 2).

Such file manipulations may be performed by a file storage and retrieval system. Such a file storage and retrieval system may be considered a trusted information source—which would, in certain embodiments, include two tracks 106 and 108.

The trusted information source may preferably be the only source for writing information to block chain 110. In certain embodiments, block chain may verify and record itinerary file 102 and medical history file 104. At 114, the information may become part of block chain.

At 120 and 122, an authenticated itinerary file and an authenticated medical file are shown. These files are preferably viewable by a third party, but not writable to by a third party, as shown at 124.

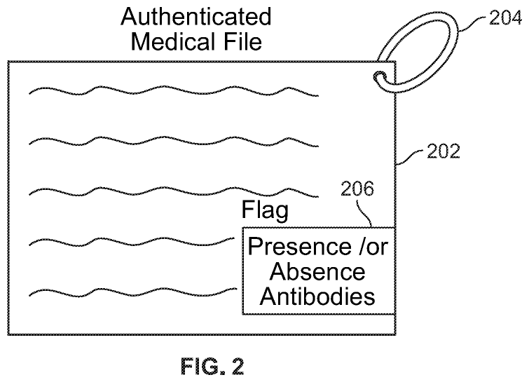

FIG. 2 shows another illustrative diagram in accordance with principles of the disclosure. Specifically, FIG. 2 shows additional detail of an authenticated medical file 202 attached to a block chain at 204. In this particular figure, a flag 206 is shown as part of the medical file. Flag 206 preferably indicates whether the user/traveler has antibodies related to a pre-determined pathogen.

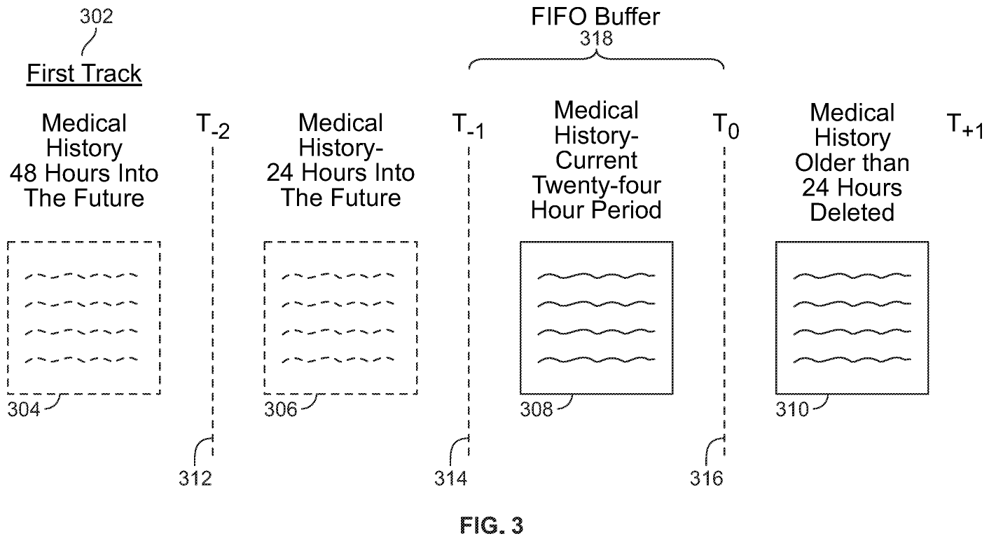

FIG. 3 shows an illustrative timing diagram along first track 302 for a user's medical history in accordance with principles of the disclosure. The medical history in the most recent twenty-four period may be shown at 308. This information may, in certain embodiments, be stored in FIFO buffer 318.

Medical history that is older than 24 hours, shown at 310, may have been deleted. It should be noted that medical history 310 is shown between T−0 and T+1.

Medical history that will happen in the next 24 hours is shown at 306. Medical history 306 is shown as occurring between 314 and 312.

Medical history that will happen between the next 24-48 hours is shown at 304. Medical history 304 is shown as occurring after T−2.

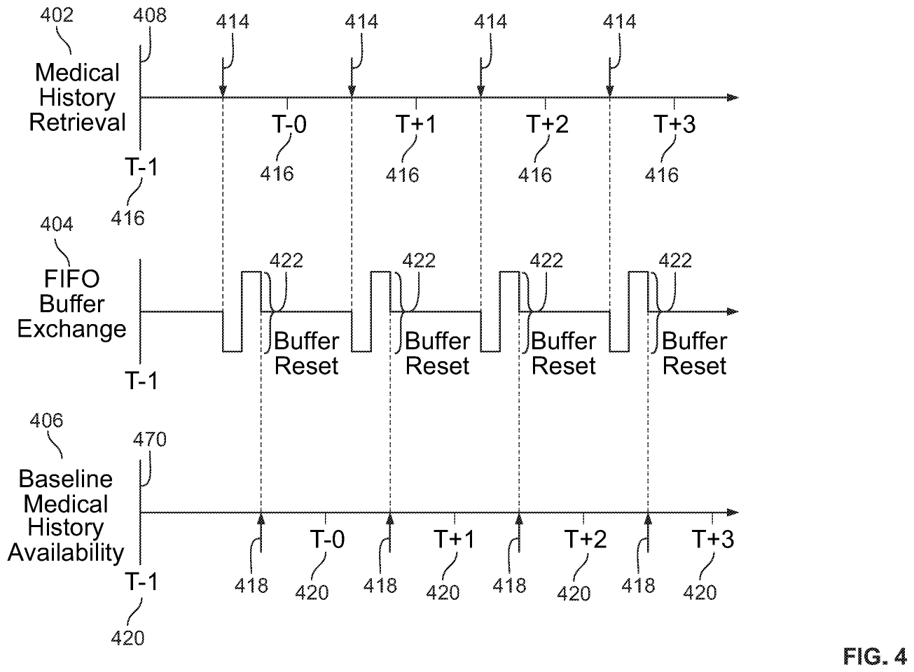

FIG. 4 shows another illustrative timing diagram in accordance with principles of the disclosure. Medical history retrieval is shown as a repeating series indicated by the element number 414. The time values are indicated by element number 416—i.e., T−1, T−0, T+1, T+2 and T+3.

FIFO buffer exchange is indicated as occurring at series 418.

Baseline medical history availability is indicated at 406. It is noted that there will be some buffer reset time period 422, which occurs periodically depending on how often the buffer is reset, during which the medical history will not be available. This is because during the buffer reset 422 it is indeterminate which is the appropriate medical history that is being accessed—e.g., a medical history from more than 24 hours in the past or a medical history that is less than 24 hours in the past.

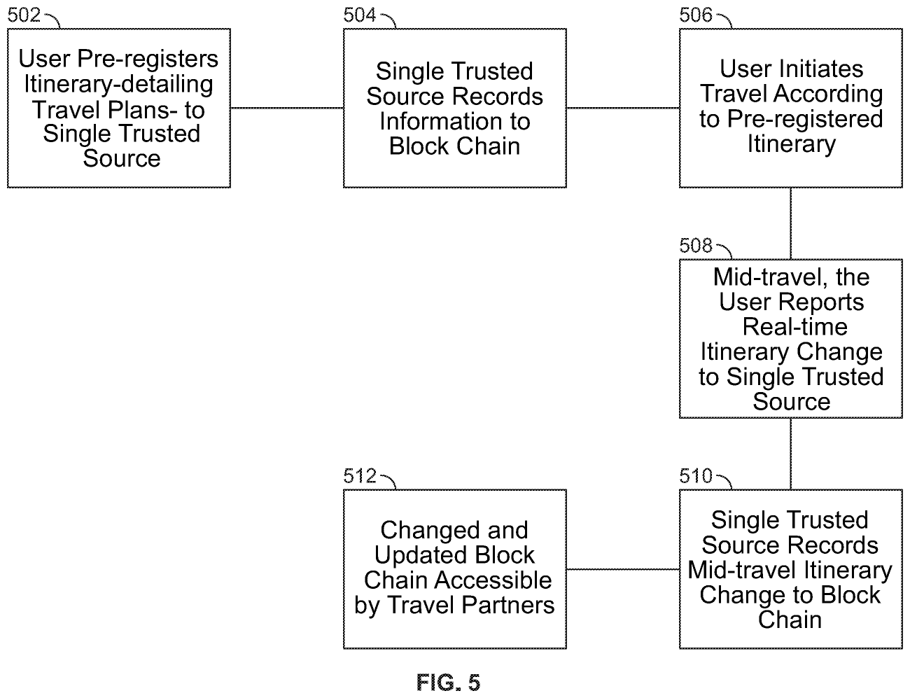

FIG. 5 shows an illustrative flow diagram in accordance with the principles of the disclosure. The flow diagram shows, at 502, that a user may pre-register his or her travel plans to a single trusted source.

Thereafter, the trusted source may record information to a block chain, as shown at step 504. Then the user may initiate travel according to the pre-registered itinerary, as shown at 506.

In the middle of the travel, the user may report a real-time itinerary change to the single trusted source, as shown at step 508. The single trusted source may then record the mid-travel itinerary change to the block chain, as shown at step 510. Thereafter, the changed and updated block chain may be made accessible to travel partners—e.g., travel service providers and/or travel companions—as shown at step 512.

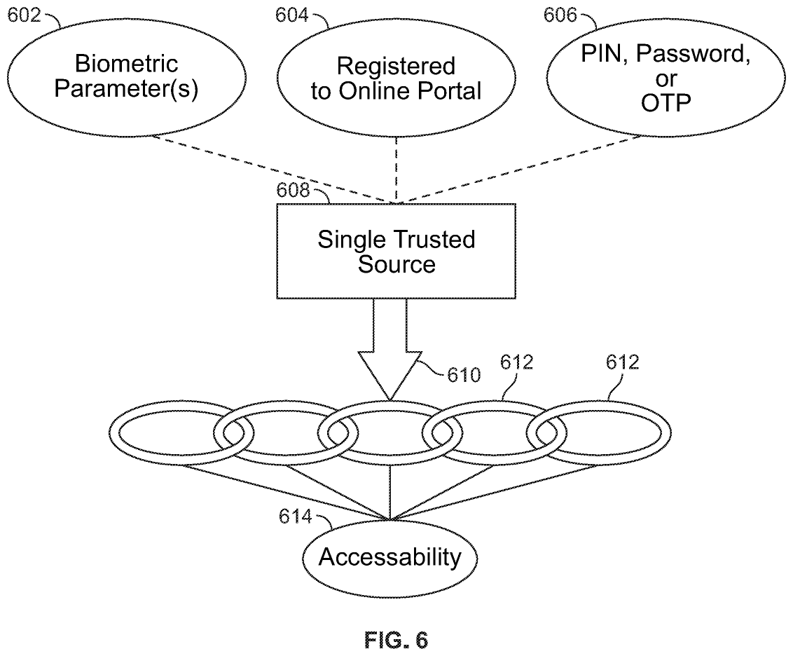

FIG. 6 shows still another illustrative diagram in accordance with the principles of the disclosure. In FIG. 6, a number of various optional inputs are shown for securing input into the single trusted source 608. Specifically, a user may secure the input, and the single trusted source 608 may only process input that is secured by, one or more biometric parameter(s), a registration to an online portal and/or a PIN, password or One Time Password (OTP). In this the information input into single trusted source 608 is protected and is accompanied by an identifiable lineage.

Thus, systems and methods involving a pre-registration system for secure travel has been provided. Persons skilled in the art will appreciate that the present invention can be practiced by other than the described embodiments, which are presented for purposes of illustration rather than of limitation.

What is claimed is:

1. A system for increasing travel security,
the system comprising:
an electronically stored user's itinerary file comprising a user's pre-registered travel itinerary, said pre-registered travel itinerary file stored on a blockchain;
an electronically stored user's medical history comprising a medical history file stored on the blockchain; and
a single trusted information source for providing information to the blockchain, the single trusted information source for providing the itinerary file and for providing the medical history file;
wherein the system comprises a dual track information storage and retrieval system, and wherein said dual track information retrieval and storage is configured to store the itinerary file and to store the recent medical history file;
wherein the dual track information storage and retrieval system comprises a first track related to the user's recent medical history file; and
wherein the dual track information storage and retrieval system comprises a second track related to the itinerary file.

2. The system of claim 1, wherein the first track further comprises a flag indicating the presence or non-presence of a pre-determined plurality of antibodies related to a pre-determined virus.

3. The system of claim 1, wherein the first track further comprises a first-in first-out (FIFO) storage system that selectively deletes a portion of the recent medical history has been stored for more than a pre-determined amount of time.

4. The system of claim 1, wherein the first track further comprises a storage system that deletes the medical history file has been stored for more than a pre-determined amount of time.

5. The system of claim 4, wherein the first track further comprises a deletion system that deletes a medical history file that has been stored for more than a pre-determined amount of time.

6. The system of claim 4, wherein the first track further comprises a deletion system that selectively deletes a medical history file that has been stored for more than a pre-determined amount of time.

7. The system of claim 4, wherein the first track further comprises a deletion system that deletes a medical history that has been stored for more than a 24-hour period.

8. The system of claim 1, wherein the second track further comprises an itinerary adjustment system that enables real-time adjustments to the itinerary in response to a communication from the single trusted information source.

9. A method for increasing travel security, the method comprising:
electronically storing on a blockchain, in response to a communication from a single trusted information source, an itinerary file comprising a user's travel itinerary;
electronically storing on the blockchain a medical history file associated with the user; and
authenticating the itinerary file and providing, using the single trusted information source, the itinerary file to the blockchain;
authenticating the medical history file and providing, using the single trusted information source, the medical history file to the blockchain;
wherein an apparatus for use with the method comprises a dual track information storage and retrieval system, and wherein said dual track information retrieval and storage system operates to store the itinerary file and the authenticated recent medical history file;
wherein the dual track information storage and retrieval system comprises a first track for storing and retrieving the medical history file; and
wherein the dual track information storage and retrieval system comprises a second track for storing and retrieving the itinerary file.

10. The method of claim 9, further comprising indicating, using an electronic flag, the presence or non-presence of greater than a pre-determined plurality of antibodies related to a pre-determined virus.

11. The method of claim 9, further comprising selectively deleting, using a first-in first-out (FIFO) storage system, at least a portion of the medical history file that has been stored for more than a pre-determined amount of time.

12. The method of claim 9, using the first track to delete a medical history file that has been stored for more than a pre-determined amount of time.

13. The method of claim 12, deleting a medical history file that has been stored for more than a pre-determined amount of time.

14. The method of claim 12, selectively deleting, using the first track, a medical history file that has been stored for more than a pre-determined amount of time.

15. The method of claim 12, deleting, using the first track, a medical history file that has been stored for more than a 24-hour period.

16. The method of claim 9, adjusting in real-time the itinerary file, using an itinerary adjustment system, in response to a communication from the single trusted information source.

17. A method for increasing travel security using a dual track information storage and retrieval system, the method comprising:
electronically storing on a blockchain an itinerary file comprising a user's travel itinerary;
electronically storing on the blockchain a medical history file associated with the user; and
authenticating the itinerary file and providing, using a single trusted information source, the itinerary file to the blockchain;
authenticating the medical history file and providing, using the single trusted information source, the medical history file to the blockchain;
wherein the dual track information retrieval and storage system operates to store the itinerary file and an authenticated recent medical history file;
wherein the dual track information storage and retrieval system utilizes a first track for storing and retrieving the user's recent medical history;
wherein the dual track information storage and retrieval system utilizes a second track for storing and retrieving the itinerary file; and
wherein the authenticating each of the itinerary file and the authenticating the medical history file comprises using the single trusted information source to verify a biometric parameter associated with the user.

18. The method of claim 17, wherein each of the authenticating the itinerary file and the authenticating the medical history file comprises using the single trusted information source to verify a one-time password associated with a user device.

19. The method of claim 17, wherein each of the authenticating the itinerary file and the authenticating the medical history file comprises using the single trusted information source to verify a registered token associated with a user device.
